(19) United States
(12) Patent Application Publication
(10) Pub. No.: US 2004/0086536 A1
(43) Pub. Date: May 6, 2004

(54) Therapeutic agents and treatment kits for hypertrophic pyloric stenosis

(76) Inventor: Akira Nagita, Okayama (JP)

Correspondence Address: BIRCH STEWART KOLASCH & BIRCH, PO BOX 747, FALLS CHURCH, VA, 22040-0747, US

(21) Appl. No.: 10/438,864

(22) Filed: May 16, 2003

(30) Foreign Application Priority Data
May 16, 2002 (JP) 2002-142037

(51) Int. Cl. A61K 9/00

(52) U.S. Cl. 424/400

(57) ABSTRACT

Hypertrophic pyloric stenosis can be treated by suppressing contraction of the pyloric muscle by using a therapeutic agent or a treatment kit which comprises atropine sulfate as an anticholinergic agent and nitroglycerin or isosorbide nitrate as an NO donor as the active ingredients.

### TECHNICAL FIELD

The present invention relates to a therapeutic agent and a treatment kit for hypertrophic pyloric stenosis.

### PRIOR ART

Hypertrophic pyloric stenosis is a disorder found in infants in which muscle at the pylorus (the outlet of stomach which is connected to the duodenum) is thickened and the pyloric lumen is narrow, whereby milk cannot pass through smoothly. Infants with hypertrophic pyloric stenosis experience projectile vomiting every time milk is fed to the infants. As a result, the infants may develop symptoms such as dehydration and decrease in body weight resulted from nutritional deficiencies. As the treatment of hypertrophic pyloric stenosis, a method of surgical incision of the thickened muscle at the pyloric part has been conventionally employed, and treatment by the oral administration of atropine sulfate has been also conducted. It is considered that hypertrophic pyloric stenosis is caused by spasm of the pyloric muscle induced by the cholinergic nervous system abnormality, and when the spasm of the pylorus persists, working hypertrophy of the pyloric muscle occurred thereby induces more strong spasm of the pylorus and finally the intestinal nerve system abnormality may be developed.

As mentioned above, it is considered that hypertrophic pyloric stenosis is caused by spasm of the pyloric muscle. In the atropine sulfate therapy, although the spasm disappears for 20 to 30 minutes after the administration of atropine sulfate, the contraction of the pyloric muscle often reappears afterward. Taking the biological half-life of atropine sulfate (about 7 hours) into consideration, the reappearance of spasm cannot be accounted for only based on the cholinergic nervous abnormality. Thus, some investigators have surmised that even if the cholinergic nerves are completely inhibited, spasm of the pylorus may occur by the compensation for the cholinergic nerves by other nervous systems.

The treatment of hypertrophic pyloric stenosis by surgery places not only heavy burdens on infantile patients but also has a fear of post-treatment infections or the like. On the other hand, it is reported that the treatment by oral administration of atropine sulfate has an effectiveness at the rate of less than 50% and therefore cannot be deemed as a surely effective treatment method; and the treatment by intravenous injection of atropine sulfate has problems such as a need of long term hospitalization, although its effectiveness is high. Accordingly, means for ensuring the treatment of hypertrophic pyloric stenosis without any burden on infantile patients has been keenly demanded.

The present invention relates to a therapeutic agent for hypertrophic pyloric stenosis. The object of the present invention is to provide a therapeutic agent and a treatment kit effective for the treatment of hypertrophic pyloric stenosis utilizing the synergistic effect of the relaxation effect on the pyloric smooth muscle by an anticholinergic agent and the relaxation effect on the pyloric smooth muscle by an NO donor.

### DISCLOSURE OF THE INVENTION

As the first embodiment of the therapeutic agent for hypertrophic pyloric stenosis according to the present invention, the therapeutic agent comprises an anticholinergic agent and an NO donor as the active ingredients.

The second embodiment of the therapeutic agent for hypertrophic pyloric stenosis according to the present invention is the therapeutic agent for hypertrophic pyloric stenosis according to the above-mentioned first embodiment, wherein the therapeutic agent is an injection.

The third embodiment of the therapeutic agent for hypertrophic pyloric stenosis according to the present invention is the therapeutic agent for hypertrophic pyloric stenosis of the above-mentioned first embodiment, wherein the therapeutic agent is a transdermal patch.

The first embodiment of the treatment kit for hypertrophic pyloric stenosis according to the present invention is a treatment kit which comprises an anticholinergic agent and an NO donor as the active ingredients.

The second embodiment of the treatment kit for hypertrophic pyloric stenosis according to the present invention is the treatment kit for hypertrophic pyloric stenosis of the above-mentioned first embodiment, wherein one of the anticholinergic agent and the NO donor is an injection, and the other is a transdermal patch.

The third embodiment of the treatment kit for hypertrophic pyloric stenosis according to the present invention is the treatment kit for hypertrophic pyloric stenosis of the above-mentioned first embodiment, wherein both of the anticholinergic agent and the NO donor are transdermal patches.

The present invention provides a method for treating hypertrophic pyloric stenosis, which comprises administering the therapeutic agent for hypertrophic pyloric stenosis of the above-mentioned first embodiment to a patient, a method for treating hypertrophic pyloric stenosis, which comprises using the treating kit for hypertrophic pyloric stenosis of the above-mentioned first embodiment to a patient, a method for treating hypertrophic pyloric stenosis, which comprises administering an anticholinergic agent intravenously and simultaneously administering an NO donor transdermally to a patient and then a method for treating hypertrophic pyloric stenosis, which comprises administering an anticholinergic agent intravenously and simultaneously administering an NO donor transdermally to a patient to remit the symptom of the hypertrophic pyloric stenosis and then administering an anticholinergic agent orally and simultaneously administering an NO donor transdermally to the patient. In the present invention, the medicine may be administered by injection or transdermally.

The anticholinergic agent of the present invention refers to an agent capable of blocking a muscarinic receptor at the parasympathetic nervous terminal. For example, atropine, scopolamine butyl bromide, butropium bromide, piperidolate hydrochloride, etomidoline, propantheline bromide, methylbenactyzium bromide, timepidium bromide, glycopyrronium bromide, N-methyl scopolamine, methyloctatropine bromide, ethylpipetanate bromide, prifinium bromide, tiemonium iodide, oxapium iodide, valethamate bromide and trospium chloride may be proposed, and atropine sulfate which is a sulfate of atropine is preferred. Atropine may be used in the form of a free body. When atropine is used in the form of a salt, it is not limited to a sulfate and other salts may be used.

In the present invention, the NO donor refers to an agent capable of supplying NO (nitrogen monoxide) in vivo when administered to a subject. The NO donor includes, for example, nitroglycerin, isosorbide nitrate, isosorbide mononitrate and amyl nitrite, and isosorbide nitrate and nitroglycerine are preferred.

In the present invention, the dosage form of the anticholinergic agent and the NO donor may be one in which the anticholinergic agent and the NO donor co-exist in a single preparation or are formulated separately in different preparations. In the case of the formulation in a single preparation, for a liquid preparation such as injection, the anticholinergic agent and the NO donor are dissolved in a single ample or vial. Of course, a pH adjusting agent such as phosphate buffer, an isotonic agent such as NaCl or mannitol which are conventionally used in injection may be used, and further, liquid preparation may be freeze-dried. As other types of dosage forms in which the two components co-exist, dosage forms suitable for transdermal administration (e.g., ointments, transdermal patches) can be mentioned. The dosage forms can be prepared in a conventional manner. In the case of dosage forms suitable for transdermal administration, the region of application is preferably a subcostal region, but is not limited thereto and any region may be used for the application.

In the present invention, the anticholinergic agent and the NO donor may be formulated separately in different dosage forms. For example, the anticholinergic agent may be formulated in an injection and the NO donor may be formulated in a preparation for transdermal application such as a transdermal patch. Of course, these two components may be formulated in a single dosage form. In the case of the formulation in different dosage forms, these two components are conveniently included in a single package. In the present invention, this form is referred to as “kit”.

The injections include intravenous injection, intramuscular injection, subcutaneous injection or the like.

The transdermal preparations include ointment, plaster, transdermal patches and the like.

In the present invention, the anticholinergic agent and the NO donor may be formulated differently from each other in the preparation form. The anticholinergic agent is preferably formulated for an intravenous injection and the NO donor is preferably formulated for a transdermal patch.

The amount to be administered of the anticholinergic agent in the present invention may vary depending on the body weights, symptoms and the like of infantile patients. For the intravenous injection of atropine sulfate, the daily dosage is generally preferably 0.04 to 0.1 mg/kg, and more preferably 0.06 to 0.1 mg/kg. The amount to be administered of the NO donor in the present invention may also depend. For a nitroglycerine patch, the daily dosage is generally 1 to 18 mg, preferably 5 to 7.5 mg. As for the anticholinergic agent and the NO donor, commercial products are available conveniently. For example, atropine sulfate and nitroglycerin are drugs described in the Japanese Pharmacopoeia and are available as chemical compounds or pharmaceutical preparations such as an injection or transdermal patches.

The therapeutic agent and the treatment kit for hypertrophic pyloric stenosis according to the present invention comprise an anticholinergic agent and an NO donor as the active ingredients and have such an effect that they enables to treat hypertrophic pyloric stenosis in a simple manner by suppressing contraction of the pyloric smooth muscle without performing a surgical operation by the relaxation effect on the smooth muscles by the anticholinergic agent and the relaxation effect on the smooth muscles by NO generated from the NO donor.

### EXAMPLES

Hereinbelow, examples of the treatment of hypertrophic pyloric stenosis using the anticholinergic agent and the NO donor of the present invention will be described. In the present treating examples, a solution for intravenous injection of atropine sulfate was used as the anticholinergic agent and a nitroglycerin patch was used as the NO donor. However, in the present invention, the preparations and the dosage forms are not limited to those used in these examples and any one may be selected properly.

After confirming that the pH and electrolytes in the blood restore in the normal ranges in individual infantile patients, a daily dosage of atropine sulfate (0.1 mg/kg/day) was divided by the number of milk feeding and the divided dosage of atropine sulfate was administered intravenously 10 minutes before each milk feeding over 3 minutes or longer. Except in the cases where abnormally strong side effects were produced, the dosage amounts for subsequent intravenous injections were not changed principally. If any side effect is produced, the daily dosage amount is gradually reduced with starting from 0.1 mg/kg/day by 0.01 mg/kg/day for every administration until the side effect is not observed, and the dosage amount at that time point is maintained. The minimum dosage amount is 0.04 mg/kg/day.

In parallel with the atropine sulfate intravenous injection, a nitroglycerin transdermal patch (Millisrol Tape (registered trademark)): 5 mg/patch: Nippon Kayaku Co., Ltd.; or Minitro Tape (registered trademark): 5 mg/patch: Nisshin-Kyorin Pharmaceutical Co., Ltd.) was used. The patch was cut in half with scissors and attached to the right hypochondriac region or the abdominal wall of the pyloric part. The tape was replaced by a new one every 12 hours or when the tape showed no adhesion. When no effect was observed even when the tape was used for 2 days, an additional one-quarter cut piece of the tape was applied every 2 days. The tape was applied at 2 pieces/day at the maximum.

To prevent the occurrence of aspiration pneumonia induced by vomiting and the induction of contraction of gastric smooth muscles and pyloric sphincters, isotonic drinking solution was fed orally as the infantile patient wanted until the vomiting subsided. When no vomiting was observed for about half a day, the solution feeding was switched to regular milk feeding (breast milk or milk powder formula) without limitation in the amount of milk intake. The efficacy of the therapeutic treatment according to the present invention was determined by confirming the disappearance of vomiting, increase in body weight, smooth passage of gastric contents through the pylorus or presence of fluid contents in the intestine under ultrasonography. In parallel with the therapeutic agents, a maintenance fluid therapy was also used for the purpose of supplying water, electrolytes and calories. The amount of the maintenance fluid therapy was reduced with increasing amount of the oral intake to control the total amount of water supplied. To prevent the overflow of milk from the mouth, the infant patient was assured to take the sitting posture or the head-erect posture. The body weight and the daily amount of milk intake were measured every day.

The possible side effects in this therapy are tachycardia or bradycardia and decreased blood pressure. For detecting these side effects, an electrocardiogram and an energometer or automated hemomanometer were attached to the infantile patient. If the pulse rate increased to 200/min or higher or decreased to 100/min or lower, the pulse pressure decreased to 40 mmHg or lower or the systolic pressure decreased to 60 mmHg or lower, then the treatment was stopped. In addition, if the systemic conditions became worse or any symptom that cannot be accounted for as a side effect was observed in the infantile patient, then the treatment was also stopped. On day 3 of the treatment, the hepatorenal functions (GPT, GOT, LDH, BUN and creatinine) were examined by the blood test. If the infantile patient experienced onset of high fever, for fear of the infection of the biliary tree, a biliary excretion-type antimicrobial agent was used in advance. In this case, the abdominal ultrasound test was performed to confirm the condition of the pancreatobiliary system. Pneumonia and sepsis (bacteremia) were also included in the items of the diagnostic determination.

After confirming the efficacy of the combination of atropine sulfate and the nitroglycerin patch for 24 hours, the atropine sulfate intravenous injection was stopped and switched to the oral administration of atropine sulfate at a dose of twice that in the intravenous injection (0.2 mg/kg/day: the figures of daily dose below the second place of decimals were omitted). Atropine sulfate in an amount given by dividing the daily dose by the number of milk feeding was administered 30 minutes before every milk feeding (6-8 times of milk feeding/day). When the infantile patient had been crying from hunger and could not wait for 30 minutes, an isotonic drink in a volume sufficient to stop the crying (30-50 ml) was given to the infantile patient after the administration of atropine sulfate. Since the gastric emptying time for an ionic drink is generally short, when the infantile patient starts to cry in the next time, then milk is fed immediately. After the observation for 1 day, if vomiting did not reappear, the intravenous fluid therapy was stopped and the infantile patient was switched to outpatient observation. After switching to the oral administration of atropine sulfate, the nitroglycerin patch was applied for additional 3 days.

If vomiting reappeared in the infantile patient after each treatment was proceeded, the procedure was returned to the previous step. If vomiting reappeared during the infantile patient was under the outpatient observation, a half piece of the nitroglycerin patch was applied on the abdominal wall of the close proximity to the pyloric part and replaced by a new one every 12 hours. To prevent the skin rashes, the position to which the patch was applied is preferably changed every time a new patch is applied. When no vomiting was observed any more, the application of the patch was continued additionally 3 days and then ended. If vomiting reappeared, this patch application procedure was repeated. The oral administration of atropine sulfate was continued for 2 weeks and then ended. By employing the therapeutic treatment method of the present invention, the duration of the intravenous injection treatment was reduced. Therefore, since this duration of oral administration (i.e., 2 weeks) might be conceivably too short, the oral administration may be continued for additionally 1 week. Alternatively, the oral administration may be performed for several days at predetermined time intervals while gradually reducing the dose amount and then ended.

Results

The results of the therapeutic efficacy by the above-mentioned therapeutic method and the therapeutic efficacy by the conventional intravenous injection method are shown in Table 1.

TABLE 1

| | Duration | |
|---|---|---|
| | of vomiting | |
| | (onset of action) | |
| | after the initiation | duration of |
| Medicine used for | of the | hospitalization |
| treatment | treatment (days) | (days) |
| atropine sulfate (iv.) | 5 ± 2 | 9 ± 2 |
| was singly used | | |
| atropine sulfate (iv.) | 2 ± 3 | 6 ± 3 |
| and nitroglycerin (iv.) | | |
| atropine sulfate (iv.) | 1 ± 1 | 4 ± 1 |
| and transdermal | | |
| nitroglycerin | | |

Nitrogen monoxide is known to be a factor capable of causing the relaxation of smooth muscles in every tissue. Since a nitrogen monoxide-producing enzyme which is sufficiently found in the pyloric muscle of healthy infants is less found in the pyloric muscle of infants with hypertrophic pyloric stenosis, some investigators have considered that the cause of hypertrophic pyloric stenosis is the deficiency or defect of the nitrogen monoxide-producing enzyme in the pyloric muscle. As shown in Table 1, when atropine sulfate which is an anticholinergic agent and nitroglycerin which is an NO donor were used in combination, the duration of vomiting after the initiation of the therapeutic treatment and the duration of hospitalization were reduced compared with those when only an atropine sulfate intravenous injection was applied, clearly demonstrating the effectiveness of the therapeutic method of the present invention. In addition, it was found that a nitroglycerin patch was more effective than an intravenous nitroglycerin injection. This is probably because the contraction of the pylorus can be suppressed by the relaxation effect on the pyloric smooth muscle by atropine sulfate based on its activity of blocking a muscarinic receptor at the parasympathetic nervous terminal and the relaxation effect on the pyloric smooth muscle by NO generated from nitroglycerin.

1. A therapeutic agent for hypertrophic pyloric stenosis comprising an anticholinergic agent and an NO donor as the active ingredients.

2. The therapeutic agent for hypertrophic pyloric stenosis according to claim 1, wherein the therapeutic agent is an injection.

3. The therapeutic agent for hypertrophic pyloric stenosis according to claim 1, wherein the therapeutic agent is a transdermal patch.

4. A treatment kit for hypertrophic pyloric stenosis comprising an anticholinergic agent and an NO donor as the active ingredients.

5. The treatment kit for hypertrophic pyloric stenosis according to claim 4, wherein one of the anticholinergic agent and the NO donor is an injection, and the other is a transdermal patch.

6. The treatment kit for hypertrophic pyloric stenosis according to claim 4, wherein both of the anticholinergic agent and the NO donor are transdermal patches.

7. A therapeutic agent for hypertrophic pyloric stenosis comprising (1) atropine sulfate and (2) nitroglycerin or isosorbide nitrate as the active ingredients.

8. A treatment kit for hypertrophic pyloric stenosis comprising (1) atropine sulfate and (2) nitroglycerin or isosorbide nitrate as the active ingredients.

9. A method for treating hypertrophic pyloric stenosis, which comprises administering the therapeutic agent according to the claim 1 to a patient.

10. A method for treating hypertrophic pyloric stenosis, which comprises using the kit according to the claim 4 to a patient.

11. A method for treating hypertrophic pyloric stenosis, which comprises administering an anticholinergic agent intravenously and simultaneously administering an NO donor transdermally to a patient.

12. A method for treating hypertrophic pyloric stenosis, which comprises administering an anticholinergic agent intravenously and simultaneously administering an NO donor transdermally to a patient to remit the symptom of the hypertrophic pyloric stenosis and then administering an anticholinergic agent orally and simultaneously administering an NO donor transdermally to the patient.
